(19) United States
(12) Patent Application Publication
(10) Pub. No.: US 2008/0097218 A1
(43) Pub. Date: Apr. 24, 2008

(54) BLOOD VESSEL PUNCTURE LOCATING APPARATUS AND METHOD

(75) Inventor: Anthony C. Vrba, Maple Grove, MN (US)

Correspondence Address: MILLER, MATTHIAS & HULL, ONE NORTH FRANKLIN STREET, SUITE 2350, CHICAGO, IL, 60606, US

(73) Assignee: BOSTON SCIENTIFIC SCIMED, INC., Maple Grove, MN (US)

(21) Appl. No.: 11/466,907

(22) Filed: Aug. 24, 2006

(51) Int. Cl. A61B 8/06

(52) U.S. Cl. 600/465

(57) ABSTRACT

An apparatus is disclosed for accurately locating a puncture in a blood vessel, the puncture being below a skin surface of a patient and accessible via an incision. The apparatus includes a probe sized for insertion through the incision and having a proximal portion including a proximal end and a distal portion including a distal tip sized for insertion through the blood vessel puncture. The probe defines a probe lumen extending from the proximal end and at least partially into the probe distal portion, the probe distal portion having a first rigidity. A core is sized for insertion into the probe lumen, the core being movable between a distal position, in which a distal end of the core is disposed within the probe distal portion, and a proximal position, in which the core is withdrawn from the probe distal portion. The core has a second rigidity greater than the first rigidity so that the distal tip has a greater rigidity with the core in the distal position and a lesser rigidity with the core in the proximal position. The probe may also include a piezo-electric transducer to assist with puncture location.

### FIELD OF THE DISCLOSURE

The present invention generally relates to methods and apparatus for performing vascular procedures, and more particularly, to devices and methods for sealing vascular puncture sites.

### BACKGROUND OF THE DISCLOSURE

Various surgical procedures are performed using percutaneous entry into a blood vessel. To facilitate cardiovascular procedures, a small gauge needle is introduced through the skin and into a target blood vessel, often the femoral artery. The needle forms a puncture through the blood vessel wall at the distal end of an incision tract that extends through the overlying tissue. A guidewire is then introduced through the bore of the needle, and the needle is withdrawn over the guidewire. For procedures requiring the use of a larger cannula, one or more dilators may be passed over the guidewire to expand the tissue opening to larger sizes. When the tissue opening is the appropriate size, an introducer sheath is advanced over the guidewire and the dilator may be removed. The sheath and guidewire are left in place to provide access during subsequent procedures.

The sheath facilitates passage of a variety of diagnostic and therapeutic instruments and devices into the vessel and its tributaries. Illustrative diagnostic procedures include angiography, intravascular ultrasonic imaging, and the like. Exemplary interventional procedures include angioplasty, atherectomy, stent and graph placement, embolization, and the like. After the selected procedure is completed, the catheters, guidewire, and introducer sheath are removed, and it is necessary to close the vascular puncture to provide hemostasis to allow healing.

Traditional methods of achieving hemostasis include the application of external pressure to the skin entry site by a nurse or physician to stem bleeding from the wound until clotting and tissue rebuilding have scaled the perforation. In some situations, this pressure must be maintained for half an hour to an hour or more, during which the patient is uncomfortably immobilized, often with sandbags and the like. With externally applied manual pressure, both patient comfort and practitioner efficiency are impaired. Additionally, a risk of hematoma exists since bleeding from the vessel may continue until sufficient clotting effects hemostasis. Also, external pressure devices such as femoral compression systems, may be unsuitable for patients with substantial amounts of subcutaneous adipose tissue since the skin surface may be a considerable distance from the vascular puncture site, by rendering skin compression inaccurate and thus less effective. Moreover, the application of excessive pressure can occlude the underlying artery, resulting in ischemia and/or thrombosis.

Even after hemostasis has apparently been achieved, the patient must remain immobile and under observation for hours to prevent dislodgement of the clot and to assure that bleeding from the puncture wound does not resume. Renewed bleeding through the tissue tract is not uncommon and can result in hematoma, pseudoaneurisms, and arteriovenous fistulas. Such complications may require blood transfusion, surgical intervention, or other corrective procedures. The risk of these complications increases with the use of larger sheath sizes, which are frequently necessary interventional procedures, and when the patient is anticoagulated with heparin or other drugs.

Various procedures have been used to promote hemostasis without relying on skin surface pressure. Some of these proposals use intraluminal plugs and are characterized by the placement of an object within the blood stream of the vessel to close the puncture. Other proposals include delivery of tissue adhesive to the perforation site. Still further proposed solutions would insert a cylindrical plug into the incision tract that would subsequently expand and seal the puncture site. All of these approaches require either introducing or leaving foreign objects in patient's body and/or inserting a tubular probe of large diameter into the tissue channel left by the catheter in order to seal the puncture.

More recently, a system for locating and therapeutically sealing a blood vessel puncture using high intensity focused ultrasound (“HIFU”) has been proposed, in which a HIFU beam is focused on the puncture site, thereby increasing the temperature at the focal region and ultimately sealing the puncture. To focus the HIFU beam on the appropriate area, the vascular puncture must first be located, such as by imaging the target site using by echo processing (e.g., a Doppler-based method). Blood vessel imaging may then be performed with the sheath and/or a rigid locator rod extended through the puncture and into the blood vessel. When removed subsequent to imaging, however, the sheath and/or locator rod may disturb the location of the blood vessel and particularly the puncture site. As a result, the measured location of the puncture site may be inaccurate, thereby causing the HIFU beam to be incorrectly focused during the vascular sealing phase of the procedure.

### SUMMARY OF THE DISCLOSURE

In view of the foregoing, an apparatus is provided for locating a puncture in a blood vessel. The apparatus includes a probe sized for insertion through an incision and having a proximal end and a distal portion including a distal tip. The probe defines a probe lumen extending from the proximal end and at least partially into the probe distal portion, the probe distal portion having a first rigidity. A core has a second rigidity greater than the first rigidity and is insertable into the probe lumen to increase the rigidity of the probe distal portion.

According to additional aspects, the apparatus may further include a piezo-electric transducer disposed in the probe distal portion to facilitate locating the puncture site, such as by detecting fluid flow or providing an electronic beacon detectable by an imaging device.

Still further, a method is provided for accurately locating a puncture in a blood vessel. The method includes providing a probe sized for insertion through an incision and having a proximal end and a distal portion including a distal tip. The probe defines a lumen extending from the proximal end and at least partially into the probe distal portion, the probe distal portion having a first rigidity. A core is provided having a second rigidity greater than the first rigidity. The core is inserted into the probe lumen and the probe and core are inserted through the incision until the probe distal end is disposed inside the blood vessel. The core is then withdrawn from the probe lumen, the location of the puncture is measured, and the probe is withdrawn from the incision

### BRIEF DESCRIPTION OF THE DRAWINGS

The foregoing aspects and many of the attendant advantages of this disclosure will become more readily appreciated as the same becomes better understood by reference to the following detailed description, when taken in conjunction with the accompanying drawings, wherein:

FIG. 1 is a side elevation view, in cross-section, of a probe with a core in a distal position in accordance with the teachings of the present disclosure;

FIG. 2 is a side elevation view, in cross-section, of the probe of FIG. 1, with the core moved toward a proximal position;

FIG. 3 is a side elevation view, in cross-section, of the probe of FIG. 1, with the core entirely withdrawn therefrom;

FIG. 4 is a side elevation view, in cross-section, of a second embodiment of a probe that forms the catheter;

FIG. 5 is a side elevation view, in cross-section, of a puncture site with access sheath prior to insertion of a probe;

FIG. 6 is a side elevation view, in cross-section, of a probe inserted through an access sheath;

FIG. 7 is a side elevation view, in cross-section, of a probe inserted through a puncture having a core moved toward a proximal position;

FIG. 8 is a side elevation view, in cross-section, of a blood vessel puncture with sheath and probe removed

### DETAILED DESCRIPTION OF THE DISCLOSURE

Apparatus and methods are disclosed for locating a puncture in a blood vessel without disturbing or changing the orientation and position of the blood vessel. The apparatus may include a probe having variable flexibility, wherein the probe is rigid during insertion into the blood vessel but is changeable to a more flexible and elastic state for removal from the blood vessel. The probe may include a flow sensor for indicating when the probe is properly positioned with respect to the blood vessel and an electronic beacon that facilitates imaging of the blood vessel structure to precisely locate the position of the puncture. The apparatus and methods are described herein in conjunction with an ultrasound device capable of imaging the vascular structure and sealing the blood vessel puncture. The disclosed embodiments are not intended to be exhaustive or limit the scope of the disclosure to the precise forms disclosed, but instead are intended to encompass any vascular device or method that would benefit from the advantages described herein.

FIGS. 1-3 illustrate a first embodiment of a device 10 for locating a puncture in a blood vessel. The blood vessel puncture is located below a skin surface of a patient and is accessible via an incision tract formed in any conventional manner. The device 10 includes a probe 12 sized for insertion through the incision tract. If a sheath is first disposed in the incision tract, the probe 12 may be sized for insertion through a lumen of the sheath. The probe 12 includes a distal portion 14 and a proximal portion 16. As used herein, the probe distal portion 14 includes at least that portion of the probe 12 that is inserted through the blood vessel puncture during a vascular procedure, as described in greater detail below. The distal portion 14 includes a distal tip 18 sized for insertion through the incision tract or, if provided, the sheath lumen, and into the blood vessel puncture, which may have a smaller diameter than the incision tract or sheath lumen. The distal tip 18 has a rounded or otherwise atraumatically shaped profile to avoid piercing or otherwise altering the blood vessel and surrounding tissue as it is manipulated within the patient. The probe 12 defines a lumen 20 that begins at a proximal end 22 of the probe 12 and terminates in the distal portion 14 near the distal tip 18.

At least the distal tip 18 of the probe 12 is formed of an elastic material. The elastic material has a relatively low rigidity (and, therefore, relatively high flexibility) which allows the distal tip 18 to bend normal to an axis of the probe 12. In a preferred embodiment, the elastic material has a stiffness approximately equal to or less than that of a standard introducer wire having a mandrel diameter of approximately 0.005-0.010 of an inch (0.13-0.25 mm). The probe may be formed of a polymer material such as polyoxymethylene (POM), polybutylene terephthalate (PBT), polyether block ester, polyether block amide (PEBA), fluorinated ethylene propylene (FEP), polyethylene (PE), polypropylene (PP), polyvinylchloride (PVC), polyurethane, polytetrafluoroethylene (PTFE), polyether-ether ketone (PEEK), polyimide, polyamide, polyphenylene sulfide (PPS), polyphenylene oxide (PPO), polysufone, nylon, perfluoro(propyl vinyl ether) (PFA), polyether-ester, polymer/metal composites, etc, or mixtures, blends or combinations thereof. One example of a suitable polyether block ester is available under the trade name ARNITEL, and one suitable example of a polyether block amide (PEBA) is available under the trade name PEBAX®, from ATOMCHEM POLYMERS, Birdsboro, Pa. One example of a suitable polyoxymethylene (POM) is Delrin™ commercially available from Dow Chemicals. In the illustrated embodiment, the entire probe 12 is formed of the thermoplastic elastomer material.

The device 10 further includes a core 30 that is insertable into the probe lumen 20 to selectively increase the rigidity of the distal tip 18. In the illustrated embodiment, the core 30 is formed generally as a rod sized for insertion into the probe lumen 20. The core 30 is movable between a distal position in which the core 30 is disposed within the probe distal portion as shown in FIG. 1, and a proximal position in which the core 30 is withdrawn from the probe distal portion 14. When in the proximal position, the core 30 may still be at least partially inserted into the probe lumen 20 as shown in FIG. 2, or may be completely withdrawn from the probe lumen 20 as illustrated in FIG. 3. The core is formed of a material having a greater rigidity of the probe distal tip 18. For example, where the probe 12 is formed of a thermoplastic elastomer, the core 30 may be formed of any more rigid material, including metal, plastic, glass, or other thermoplastic elastomers having a higher hardness than the probe thermoplastic elastomer material. Accordingly, when the core 30 is disposed within the probe 12 and in the distal position, the distal tip is stiffened. Conversely, when the core 30 is in the proximal position, the probe distal tip 18 is in a more flexible state.

The probe 12 may further include a piezo-electric transducer that may measure fluid flow, assist with puncture location imaging, or both. As illustrated in FIGS. 1-3, the piezo-electric transducer 34 has an annular shape and may be molded inside the probe 12. A lead line 36 extends from the piezo-electric transducer and extends externally from the proximal end 22 of the probe 12 for connection to an electrical device that may be capable of receiving and/or sending electrical signals. The piezo-electric transducer 34 may be used as a fluid flow sensor to assist in positioning the probe with respect to the blood vessel puncture. Specifically, the piezo-electric transducer 34 may detect blood flow as the probe 12 is inserted through the incision tract, thereby indicating that the piezo-electric transducer 34 is adjacent to the puncture and the distal tip 18 is disposed inside the blood vessel.

Additionally or alternatively, the piezo-electric transducer 34 may also provide an electronic beacon for use during blood vessel imaging. The piezo-electric transducer 34 may generate a signal that is detectable by ultrasound or other methods of imaging vascular and tissue structure to provide a more definitive and clear reference point indicating the location of the puncture. When the probe 12 is positioned within the incision tract such that the piezo-electric transducer 34 is coincident with the blood vessel puncture, the location of the blood vessel puncture may be more precisely identified. The aforementioned fluid flow sensing function may assist in positioning the probe 12 so that the piezo-electric transducer 34 is coincident with the blood vessel puncture.

While the piezo-electric transducer 34 is described as having two functions, it will be appreciated that it may perform only one of those functions without departing from the scope of this disclosure. Various vascular sealing methods, many of which do not use ultrasound imaging, require the practitioner to identify the location of the puncture, or at least the depth below the skin surface at which a blood vessel puncture is located. A fluid flow sensor positioned at a known location on the probe 12 will allow the practitioner to at least measure the depth of the blood vessel puncture below the skin surface. For example, the probe may be inserted through the incision tract until the sensor detects fluid flow, and the practitioner may mark or otherwise indicate on an exterior of the probe a location of the skin surface. When the probe is subsequently withdrawn, the distance between the skin surface location and the fluid flow sensor can be measured to provide an approximate depth of the blood vessel puncture below the skin surface.

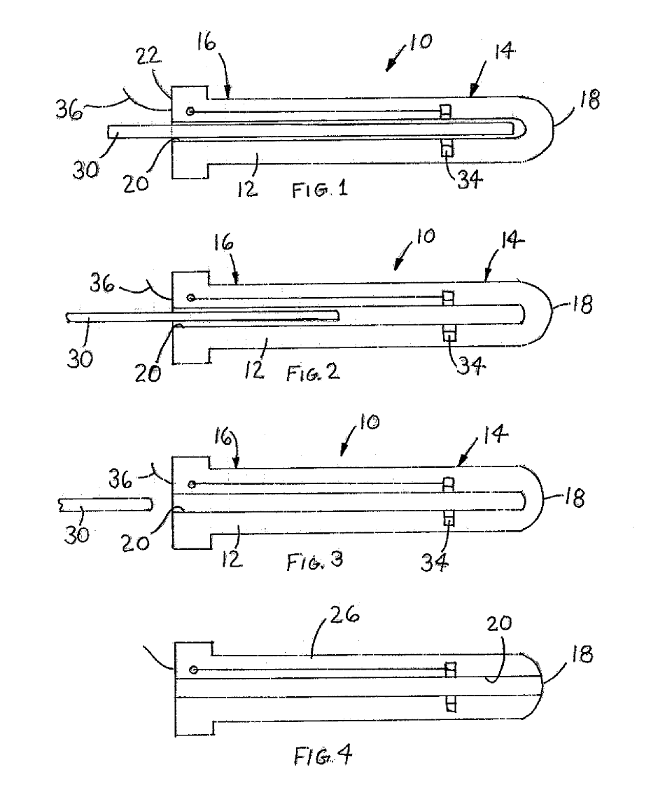

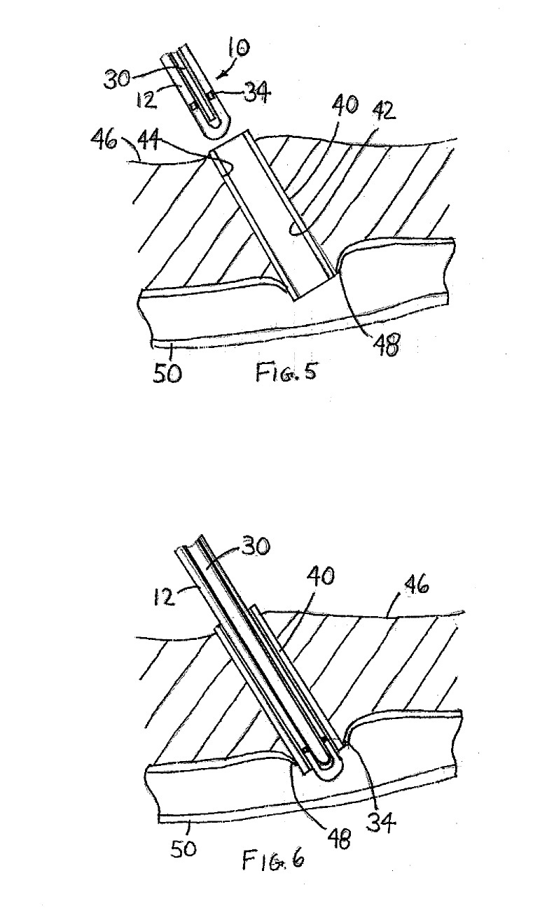

The probe 12 may be provided as an obturator, characterized by a closed distal tip as shown in FIGS. 1-3. Alternatively, the probe lumen 20 may extend entirely though the probe distal tip 18 to form a catheter like probe 26 as illustrated in FIG. 4. In either case, the probe distal tip is formed of elastic material that is stiffened by the core 30 when in the distal position.

A method of using the probe 12 is illustrated in FIGS. 5-9. In the embodiment illustrated in FIG. 5, a sheath 40 defining a lumen 42 is positioned within an incision tract 44 formed in a patient. The incision tract 44 provides access from a skin surface 46 to a puncture 48 formed in a blood vessel 50. The sheath 40, which may have been positioned in the incision tract 44 for use during a vascular procedure, may remain in place to assist with the insertion of the probe 12. In this embodiment, therefore, the probe 12 has an outer profile sized for insertion through the sheath lumen 42

As shown in FIG. 5, the probe 12 with core 30 in the distal position is axially aligned with the sheath lumen 42 in preparation for insertion through the sheath 40. The probe 12 and core 30 may be advanced through the sheath lumen 42 until the probe distal tip 18 is disposed inside the blood vessel 50, as illustrated in FIG. 6. As the probe 12 is advanced through the sheath 40, piezo-electric transducer 34 may be used to detect blood flow, thereby indicating when the probe 12 is properly positioned relative to the blood vessel puncture. Up to this point in the process, it is beneficial for the probe distal tip 18 to be relatively rigid to withstand any forces that resist insertion of the probe into the tract 44, particularly in cases where the sheath 40 is not present.

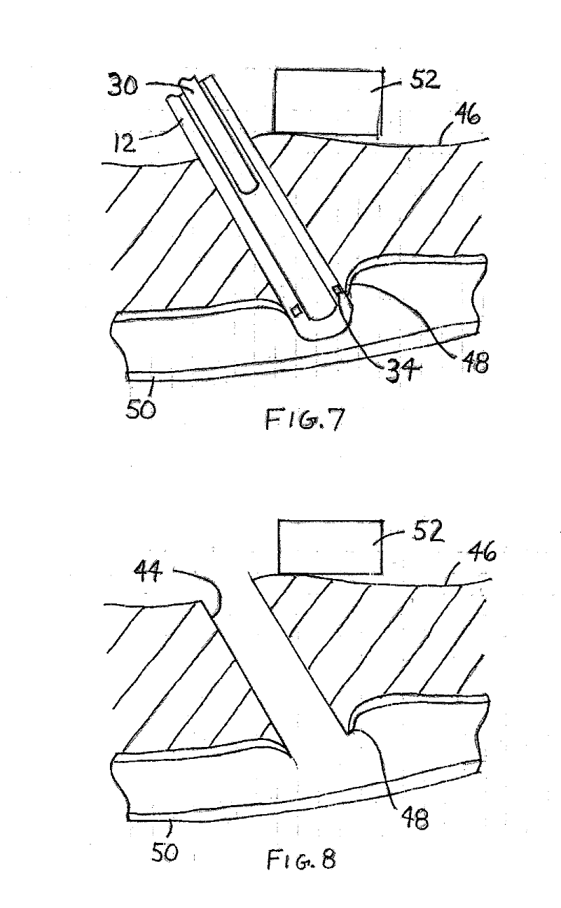

With the probe 12 properly positioned, the sheath 40 may be removed from the incision tract 44. This may be accomplished by applying a force in the distal direction to the probe 12 while the sheath 40 is proximately removed from the incision tract 44, so that the probe 12 remains in substantially the same position as it was prior to sheath removal, as illustrated in FIG. 7. Again, it is desirable for the probe distal tip 18 to be relatively rigid while the sheath 40 is removed, thereby to maintain the probe 12 in a substantially stationary position.

After the sheath 40 is removed, the core 30 may be withdrawn to the proximal position to increase the flexibility of the probe distal tip 18, as illustrated in FIG. 7. Increased flexibility is desirable to accurately locate the puncture site. While various location methods may be employed, FIG. 7 illustrates an imaging device 52, such as an ultrasound transceiver, which is capable of mapping the positions of structures located below the skin surface. One example of an ultrasound device is disclosed in U.S. Pat. No. 6,656,136 to Weng et al, the disclosure of which is incorporated herein by reference. As noted above, the piezo-electric transducer 34 located in the probe distal tip 18 may assist during mapping by providing an electronic beacon that is readily detectable by the imaging device and is clearly identifiable on the imaging display. The location of the beacon corresponds to the location of the blood vessel puncture 48, so that vascular sealing operations may be directed to the appropriate area.

Once the blood vessel 50 and puncture 48 have been located, the probe 12 may be removed from the blood vessel puncture 50 and incision tract 44, as illustrated in FIG. 8. Because the distal tip 18 is placed in a relatively flexible state prior to removal, the blood vessel 50 will remain substantially in its undisturbed, initial orientation and location. As a result, a subsequent sealing procedure may more reliably use the previously collected mapping information to target the appropriate area for sealing.

While the foregoing was written with reference to specific examples and embodiments, it is to be under stood that the scope of the invention is not to be limited thereby, but rather they are provided to satisfy best mode and enablement requirements while providing support for any and all claims which may issue herefrom.

What is claimed is:

1. Apparatus for locating a puncture in a blood vessel, comprising:
a probe sized for insertion through an incision and having a proximal end and a distal portion including a distal tip, the probe defining a probe lumen extending from the proximal end and at least partially into the probe distal portion, the probe distal portion having a first rigidity; and
a core having a second rigidity greater than the first rigidity, the core being insertable into the probe lumen to increase the rigidity of the probe distal portion.

2. The apparatus of claim 1, in which the probe comprises a catheter and the probe lumen extends through the distal tip.

3. The apparatus of claim 1, in which the probe comprises an obturator.

4. The apparatus of claim 1, in which the probe distal tip is formed with an atraumatic profile.

5. The apparatus of claim 1, in which the core is adapted to be completely withdrawn from the probe.

6. The apparatus of claim 1, in which a sheath is configured for insertion through the incision and into a blood vessel puncture, the sheath defining a lumen sized to receive the probe.

7. The apparatus of claim 1, further comprising a fluid flow sensor disposed in the probe distal portion.

8. The apparatus of claim 7, in which the fluid flow sensor comprises a piezo-electric transducer.

9. The apparatus of claim 1, further comprising an electronic beacon disposed in the probe distal portion.

10. The apparatus of claim 9, in which the electronic beacon comprises a piezo-electric transducer.

11. Apparatus for locating a puncture in a blood vessel, the puncture being below a skin surface of a patient and accessible via an incision the apparatus comprising:
a probe sized for insertion through the incision and having a proximal portion including a proximal end and a distal portion including a distal tip sized for insertion through the blood vessel puncture, the probe defining a probe lumen extending from the proximal end and at least partially into the probe distal portion, the probe distal portion having a first rigidity;
a piezo-electric transducer disposed in the probe distal portion; and
a core sized for insertion into the probe lumen, the core being movable between a distal position, in which a distal end of the core is disposed within the probe distal portion, and a proximal position, in which the core is withdrawn firm the probe distal portion, the core having a second rigidity greater than the first rigidity so that the distal tip has a greater rigidity with the core in the distal position and a lesser rigidity with the core in the proximal position.

12. The apparatus of claim 11, in which the piezo-electric transducer measures fluid flow.

13. The apparatus of claim 11, in which the piezo-electric transducer provides an electronic beacon identifiable by an ultrasound device.

14. The apparatus of claim 11, in which the probe distal tip is formed with an atraumatic profile.

15. The apparatus of claim 11, in which the core is completely withdrawn from the probe in the core proximal position.

16. The apparatus of claim 11, in which a sheath is inserted through the insertion and into the blood vessel puncture, the sheath defining a lumen, and in which the probe is sized for insertion through the sheath lumen.

17. A method of locating a puncture in a blood vessel, comprising:
providing a probe sized for insertion through an incision and having a proximal end and a distal portion including a distal tip, the probe defining a probe lumen extending from a proximal end and at least partially into the probe distal portion, the probe distal portion having a first rigidity;
providing a core having a second rigidity greater than the first rigidity;
inserting the core into the probe lumen;
inserting the probe and core through the incision until the probe distal end is disposed inside a blood vessel;
withdrawing the core from the probe lumen;
measuring the location of the puncture; and
withdrawing the probe from the incision.

18. The method of claim 17, in which a sheath is inserted into the incision and has a lumen sized to receive the probe, the method further comprising, prior to withdrawing the core, holding the probe and core relatively stationary while withdrawing the sheath from the incision.

19. The method of claim 17, in which a fluid flow sensor is disposed in the probe distal portion, and in which the insertion of the probe and core through the incision includes monitoring the fluid flow sensor to determine when the probe distal end is disposed inside the blood vessel.

20. The method of claim 17, in which an electronic beacon is disposed in the probe distal portion, and in which measuring the location of the puncture includes sensing the position of the electronic beacon with the probe distal end disposed in the blood vessel.
